SEQUENCE LISTING (9 sequences)

SEQ ID NO: 1 - DNA, 34 bp, Artificial Sequence
  chemically synthesized sequence
ctagtgctagccaccatggagctagttctagagc

SEQ ID NO: 2 - DNA, 34 bp, Artificial Sequence
  chemically synthesized sequence
ggccgctctagaactagctccatggtggctagca

SEQ ID NO: 3 - DNA, 9 bp, Artificial Sequence
  chemically synthesized sequence
gccaccatg

SEQ ID NO: 4 - DNA, 18 bp, Artificial Sequence
  chemically synthesized sequence
ccgcgcgctccccgctgc

SEQ ID NO: 5 - DNA, 20 bp, Artificial Sequence
  chemically synthesized sequence
ggtttgtccaaactcatcaa

SEQ ID NO: 6 - RNA, 11 nt, Artificial Sequence
  chemically synthesized sequence
ggcuagccacc

SEQ ID NO: 7 - DNA, 33 bp, Artificial Sequence
  chemically synthesized sequence
gtagccgcgcttacccttacccttaccctaacc

SEQ ID NO: 8 - DNA, 129 bp, Artificial Sequence
  chemically synthesized sequence
gagctccaccgcggtggcggccgctctagaactagctccatggtggctagcactagtggatcccccgggctgcaggaattcgatatcaagcttatcgataccgtcgacctcgagggggggcccggtacc

SEQ ID NO: 9 - DNA, 129 bp, Artificial Sequence
  chemically synthesized sequence; complementary sequence of sequence 8
ggtaccgggccccccctcgaggtcgacggtatcgataagcttgatatcgaattcctgcagcccgggggatccactagtgctagccaccatggagctagttctagagcggccgccaccgcggtggagctc

(19) United States
(12) Patent Application Publication
(10) Pub. No.: US 2004/0235175 A1
(43) Pub. Date: Nov. 25, 2004

(54) Method for transfection of rna using electrical pulses

(76) Inventors: Gustav Gaudernack, Oslo (NO); Stein Saeboe-Larssen, Oslo (NO)

Correspondence Address: FITZPATRICK CELLA HARPER & SCINTO, 30 ROCKEFELLER PLAZA, NEW YORK, NY, 10112, US

(21) Appl. No.: 10/476,640

(22) PCT Filed: Apr. 22, 2002
(86) PCT No.: PCT/NO02/00150

(30) Foreign Application Priority Data
May 4, 2001 (GB) 0111015.4

(51) Int. Cl. C12N 15/87; C12N 5/08

(52) U.S. Cl. 435/459; 435/372

(57) ABSTRACT

The present invention relates to a method of inserting RNA into cells. In this method, cells are transfected with RNA using electroporation in order to achieve high transfection efficiency. The method is useful, inter alia, in providing cells to be used in cell-based therapies, e.g. in preparing cells useful as anti-cancer vaccines. Preferably, the RNA has a 5′ cap and a 3′ poly (A) tail.

The present invention relates to a method of inserting genetic material into cells.

The introduction of genetic material into cells is of fundamental importance to developments in modern biology and medicine, and has provided much of our knowledge of gene function and regulation. In nearly all cases DNA has been used for transfection purposes because of its inherent stability and its ability to integrate into the host genome to produce stable transfectants. A wide variety of methods are available to introduce genetic material into cells. These include simple manipulations such as mixing DNA with calcium phosphate, DEAE-dextran, polylysine, or carrier proteins. Other methods involve microinjection, protoplast fusion, liposomes, gene-gun delivery, and viral vectors, to mention some.

According to the present invention there is provided a method comprising using one or more electrical pulses to introduce RNA into a cell.

Although electroporation techniques are known, they have concentrated upon using DNA.

RNA-based transfection has focused upon other techniques. For example, strategies for mRNA-mediated transfection have been developed using RNA/liposome complexes (Malone, R. W. et al., 1989, Proc. Natl. Acad. Sci. USA. 86: 6077-6081; Glenn, J. S. et al., 1993, Methods Enzymol. 221: 327-339; Lu, D. et al., 1994, Cancer Gene Ther. 1: 245-252) or simply by incubating cells and mRNA together (Boczkowski, D. et al., 1996, J. Exp. Med. 184: 465-472; Boczkowski, D. et al., 2000, Cancer Res. 60: 1028-1034).

By utilising the method of the present invention, surprisingly high transfection efficiencies can be achieved. Transfection efficiencies achievable using the method of the present invention can sometimes be several hundred times greater than those achieved using certain other RNA-based techniques. The method of the present invention can also be used for the transfection of primary cells, such as dendritic cells (DCs), for which direct measurements of ectopical expression after mRNA transfection have not previously been demonstrated (see Mitchell D. A. and Nair S. K., 2000, J Clin Invest 106: 1065-1069). The present invention therefore represents a major breakthrough in the field of transfection.

Preferably the method of the present invention uses RNA with a poly(A) tail. The poly(A) tail is desirably at least 10, at least 20, at least 50, or at least 70 nucleotides long.

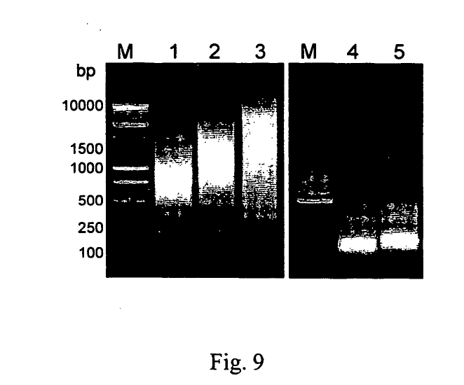

In some cases the poly(A) tail may even be over 100, over 200, over 500, or over 1000 nucleotides long. Long poly(A-T) chains are a preferred aspect of the present invention and can be achieved by using high temperature PCR with a thermostable polymerase enzyme (see Examples and FIG. 9). For example, long poly(A-T) chains can be generated in a PCR reaction containing oligo d(A) and oligo d(T) oligonucleotides, a thermostable DNA polymerase such as Pfu (Stratagene), dATP and dTTP deoxynucleotides, and additional buffer components required by the enzyme (usually supplied with the enzyme). The PCR is run on a thermal cycler, for example a PTC-200 (MJ Research, Waltham, Mass.), using a suitable temperature profile which is repeated for 1-100 cycles, more preferably 20-30 cycles. A profile consisting of 20° C. for 1 seconds followed by 70° C. for 10 seconds, may be used, but other profiles may also produce good results (FIG. 9). By using such a method the present inventors have been able to obtain very long poly(A-T) chains of up to 10000 nucleotides long. Non-thermostable DNA polymerases, such as the large Klenow fragment of E. coli DNA polymerase I, can also be used with the method, and produce shorter poly(A-T) chains mainly in the range 100-250 nucleotides (see Examples and FIG. 9). Other methods for production of poly(A-T) chains, such as chemical synthesis, or coupling of shorter fragments using a ligase enzyme, may also be used. Poly(A-T) chains may also be obtained from a suitable cDNA source.

Desirably the RNA has a 5′ cap. The 5′ cap may, for example, be m⁷G(5′)ppp(5′)G, m⁷G(5′)ppp(5′)A, G(5′)ppp(5′)G or G(5′)ppp(5′)A cap analogs, which are available on the commercial market, for example from NEB Inc., Beverly, Mass. Other chemical structures with the ability to promote stability and/or translation efficiency may also be used.

In a most preferred embodiment of the present invention the RNA has both a 5′ cap and a 3′ poly(A) tail.

The RNA may be mRNA, but this is not essential. Other molecules can be provided with a 5′ cap and/or a poly(A) tail by standard techniques. For example, antisense RNA that has the ability to hybridise to and abolish the function of other RNAs may be synthesised with a 5′ cap and/or a poly(A) tail and may be used in the present invention.

The RNA may optionally also have one or more additional features. For example, stabilising elements, such as the untranslated regions from the -globin RNA, may be present. It may also be advantageous that the mRNA lacks a 3′ UTR, so that the stop codon at the end of the open reading frame is fused directly to the poly(A) tail.

The present invention can utilise electroporation techniques to provide electrical pulses. The results provided in the examples were obtained using a square-wave electroporator (see Materials and Methods). Square wave electroporation is preferred for the present invention, although other techniques can be used.

The method of the present invention is notable in that it provided high-level transfection of all cell types that were tested, including primary cells (prepared directly from the tissues of an organism) such as DCs, lymphocytes and CD34+ stem cells, and also Epstein-Barr-virus transformed B cells (EB) and several cancer cell lines.

There is nothing in the literature to indicate that such high levels of transfection can be achieved using electroporation of RNA. As indicated above electroporation of nucleic acids has concentrated upon DNA. There is an isolated article in which glacZgₙ RNA is electroporated into HeLa-K^{b} cells for experimental purposes (Hoerr et al, Eur. J. Immunol. 30: 1-7 [2000]). The electroporation of glacZgₙ RNA into HeLa-K^{b} cells as disclosed in this article is expressly disclaimed from the scope of the present invention. It is notable that there is no discussion in the article of the transfection levels obtainable using electroporation. Indeed the article focuses upon vaccination protocols involving the direct injection of protamine-condensed RNA (in encapsulated or non-encapsulated form) or of naked RNA. It is indicated that protamine-condensed RNA is advantageous in that it could be used at very low levels (compared to DNA) to stimulate a CTL response. None of the vaccination protocols described involve electroporation. Cells can be efficiently transfected using the method of the present invention for a wide range of different instrument settings, involving variations in voltage, pulse length and number of pulses applied. For instance, optimal transfection of DCs (dendritic cells) can be achieved by applying a voltage (as reported by the instrument) in the range 1.5-3 kV/cm (kilovolt/cm) for 0.15-0.25 milliseconds (ms), but considerable transfection is also obtained both at higher voltages combined with shorter pulses, and lower voltages combined with longer pulses. For CD34+ stem cells the optimal range involves higher voltage combined with shorter pulse length (2.2-4 kV/cm for 0.03-0.1 ms), whereas for EB cells and cancer cell lines it is advantageous to utilise a lower voltage and a longer duration of the pulse (0.5-1.5 kV/cm for 0.43-40 ms).

For transfection of the primary cells mentioned above (DCs, lymphocytes and CD34+ stem cells), long-lasting pulses (several milliseconds or more) in combination with low voltage was less efficient and/or deleterious to the cells. The optimal conditions for electroporation of these cell types are thus considerably different from what have been established for cell lines and many other cell types, which generally involve a combination of low voltage and a long-lasting pulse. (A compilation of current protocols can be obtained from BTX at http://www.btxonline.com/btx/index.html.)

In any event, a skilled person will be able to determine appropriate electroporation conditions for any given cell and RNA molecules by routine experimentation. It is therefore not intended that the conditions discussed above should be limiting. Thus pulse lengths may vary greatly (e.g. from 0.0001 to 100000 milliseconds, more preferably from 0.01 to 1000 milliseconds).

Voltages may also vary greatly (e.g. from 0.001 to 1000 kV/cm, more preferably from 0.1 to 10 kV/cm). Cells can be immersed in various types of buffer/medium during electroporation. Commercially available growth media such as RPMI, IMDM, X-VIVO and PBS, to mention some, have produced good results when used with the present invention. For many cell types, transfection efficiencies and/or survival rates can be improved by using specially composited buffers. For example, the buffer may contain lower concentration of salts, and/or potassium salts may be used instead of sodium salts. For any given cell type, an optimal electroporation buffer can be determined by routine experimentation. The temperature of cells/buffer used for electroporation is preferably below 43° C., more preferably 0-4° C.

The methods of the present invention have a vast range of applications. Some of these are discussed below.

The last two decades have seen intensive efforts to clone and characterise genes from pathogenic sources and tumour tissues. Based on this knowledge, new and important approaches for medical treatment and vaccination regimes have emerged using such genes and mutations as antigens to induce protective immune responses. Because genetic vaccines are relatively inexpensive and easy to manufacture, and can be administered directly by injection into skin, their immunogenicity and efficacy have been analysed in a large number of systems. Studies have rapidly moved from small laboratory animals to primates and clinical trials are currently being conducted for diseases such as malaria, HIV-infection, and cancer. However, the efficacy of genetic vaccines in many systems has not proven to be satisfactory, in particular in organisms with high body mass (reviewed in Manickan E. et al., 1997, Crit. Rev. Immunol. 17: 139-154).

The recognition of dendritic cells (DCs) as the most potent antigen-presenting cells for inducing T-cell mediated immune responses has shifted the emphasis in vaccine development (reviewed in Banchereau J. and Steinman R. M., 1998, Nature 392: 245-252). The rationale is that DCs loaded with appropriate antigens will migrate to regional lymph nodes to activate antigen-specific T cells. Large numbers of DCs can easily be generated from blood by culturing adherent mononuclear cells in the presence of cytokines (Romani, N. et al., 1995, J. Exp. Med. 180: 83-93), and many studies have documented priming of T-cell responses in mice after vaccination with such DCs loaded with antigens. Recently vaccination of cancer patients with antigen pulsed dendritic cells have resulted in strong immune responses that is correlated with clinical benefit in different groups of cancer patients (Nestle, F. O. et al., 1998, Nat Med. 4: 328-332; Schadendorf, D. and Nestle, F. O., 2001, Recent Results Cancer Res. 158: 236-248. Review.; Yu, J. S. et al., 2001, Cancer Res. 61: 842-847).

The present invention allows transfection of DCs (or of other cells) to be achieved so as to lead to the expression of only those proteins for which an immune response is to be targeted. Expression of proteins other than the relevant antigens may interfere with generating the intended response due to immunodominance (Pion, S. et al., 1999, Blood. 93: 952-962) and pre-existing immunity. This is in particular relevant to viral vectors, which have been most extensively used for transfection of primary cells in vivo and in vitro because of their high efficiency of transfection.

The present invention is also advantageous in that it circumvents potential problems involving transcriptional regulation by providing RNA directly, which has easy access to the cytoplasmic translation machinery upon entry into the cell. Furthermore, RNA is often a safer alternative than DNA due to its limited ability to cause permanent genetic mutations in the host.

RNA can be isolated directly from cell samples, such as tumour biopsies or it may be synthesised, e.g. by chemical or gene-cloning methods.

RNA-based electroporation also provides a convenient method for direct transfection of tumour derived mRNA into DCs (or other cells) for stimulation of cellular immune responses (e.g. T cell responses), and may eliminate the need for prior amplification steps as undertaken by others (Boczkowski, D. et al., 2000, Cancer Res. 60: 1028-1034).

RNA-based transfection can be used to induce cell interactions. For instance, DCs transfected with hTERT/pCIpA₁₀₂ mRNA can be used to induce hTERT-specific cytotoxic T lymphocytes (see the examples). Immunotherapy based on this strategy can in principle be used with any protein-encoding RNA. A significant advantage of the method is the ability to load cells with a single protein at a time, as opposed to virus-mediated transfection.

Cells can be transfected using the present invention to study regulation of cellular processes. For example, mRNA encoding a transcription factor such as MYC or FOS may be electrotransfected into cells and the effects on phenotype and/or changes in expression of other proteins may be monitored using standard techniques, for example to improve understanding of gene regulation and function, and regulatory cascades. In a similar manner, mRNA encoding mutated forms of cellular proteins, such as V-MYC, V-FOS or RAS-12Cys, may be electotransfected into cells to study the effects of such mutations on cellular processes and cancer development.

The invention will now be described by way of example only with reference to the accompanying figures, wherein:

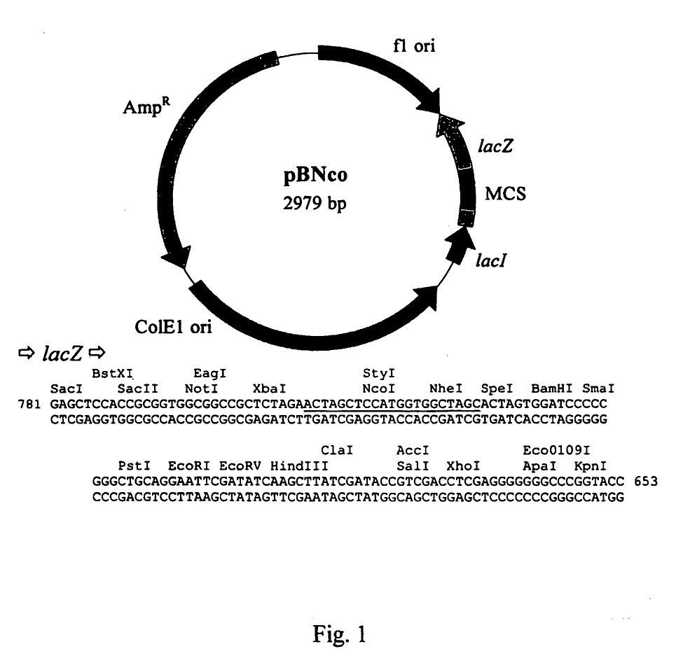

FIG. 1 is a schematic drawing of the pBNco shuttle vector. The nucleotide sequence of the multiple cloning site (MCS), which forms part of the lacZ open reading flame, is shown on the right side with unique restriction cut sites annotated above the sequence. pBNco was constructed by the addition of Nco I and Nhe I cut sites to the MCS of pBluescript SK(−). The inserted sequence harbouring these cut sites is underlined in the MCS-sequence panel. Other annotations used in the drawing are: lacI, lac promoter; Co1E1 ori, E. coli origin of replication; f1 ori, f1 phage origin of replication; Amp^{R}, -lactamase gene.

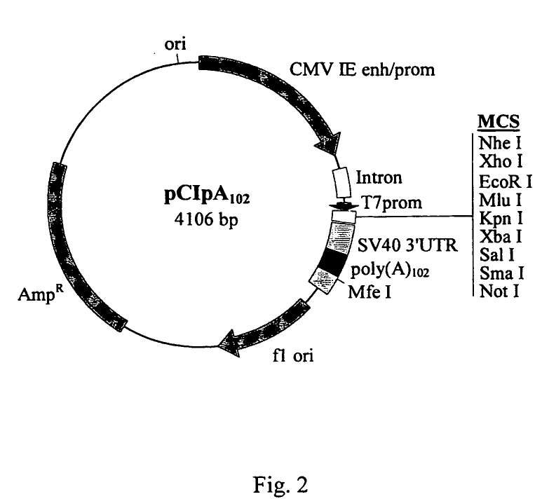

FIG. 2 is a schematic drawing of the pCIpA₁₀₂ expression vector. pCIpA₁₀₂ was made by modification of the pCI expression vector to facilitate production of polyadenylated mRNA by in vitro transcription. A 100-bp poly(A-T) fragment was inserted into the Hpa I cut site located in the SV40 late polyadenylation signal in pCI, and is represented by a black box. The Mfe I cut site located immediately downstream of the poly(A) region is used to linearize the plasmid prior to in vitro trascription. The annotations used are: CMV IE enh/prom, CMV immediate-early enhancer/promoter, intron, chimeric intron; T7prom, T7 promoter; MHC, multiple cloning site; SV40 3′UTR, 5′-most region (131 bp) of the SV40 late polyadenylation signal; poly(A)₁₀₂, 102-bp poly(A) region; f1 ori, f1 phage origin of replication; ori, E. coli origin of replication; Amp^{R}, -lactamase gene.

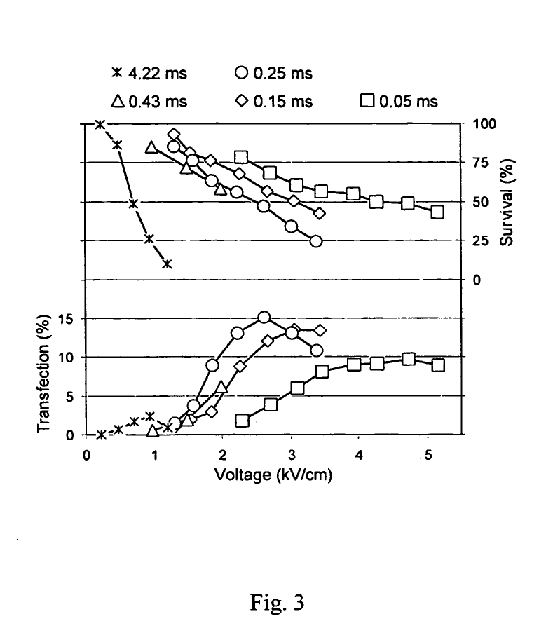

FIG. 3 is a graph illustrating square-wave electroporation of DCs with DNA at different instrument settings. DCs were electroporated with the EGFP/pCI DNA construct at different combinations of voltage and pulse length in order to identify the optimal conditions for transfection. The different pulse lengths used are indicated at the top, and the specified voltage on the x-axis is defined as the actual voltage reported by the instrument divided by 0.2 cm which is the distance between the electrodes in the cuvette used for electroporation. Transfection efficiency (lower panel) and survival rate (upper panel) is shown as percentage of the total number off cells in each sample.

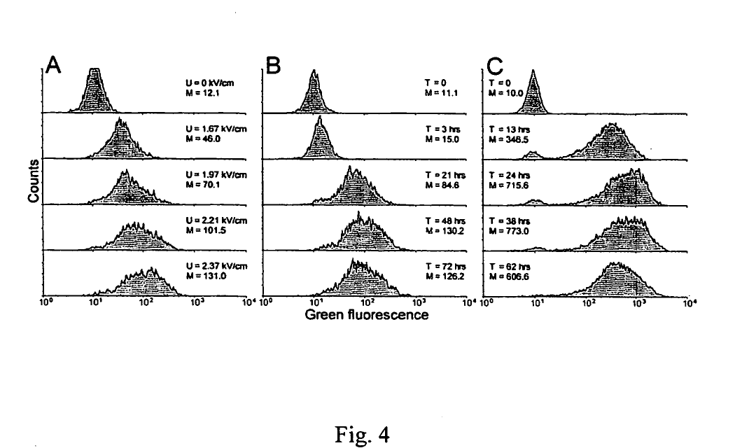

FIG. 4 is a histogram representation of DCs transfected with EGFP/pCIpA₁₀₂ mRNA by square-wave electroporation. Cells were electroporated for 0.25 milliseconds and: A, different voltage settings using mRNA produced as described in the Ribomax-T7 kit manual; B-C, ˜2.4 kV/cm and different incubation periods between transfection and analysis using mRNA produced as described in the Ribomax-T7 kit manual (panel B) or by using a modified protocol with increased concentrations of rGTP and cap analogs as described in Materials and Methods (panel C). The experiments were performed twice with practically identical results. U, voltage reported by the instrument; T, incubation period after transfection; M, mean fluorescence of living cells.

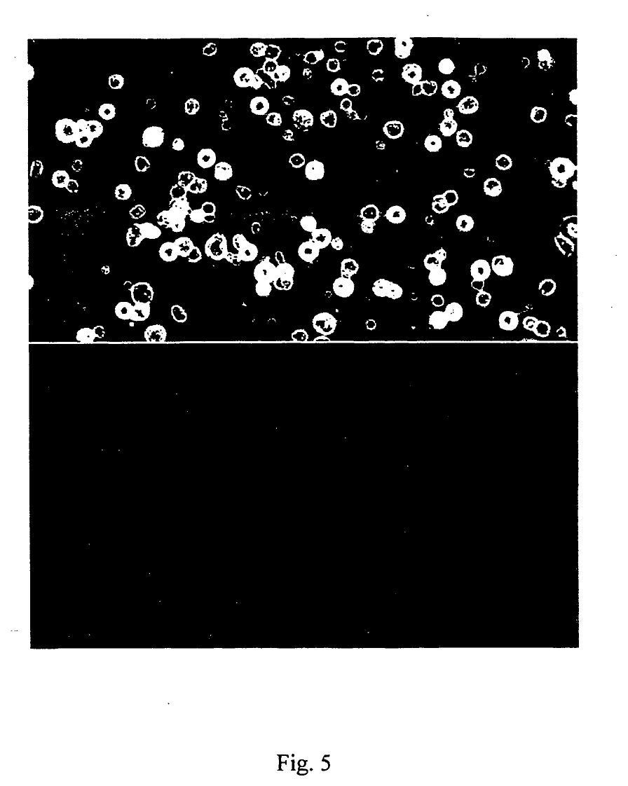

FIG. 5 shows microscopy pictures of DCs five days after transfection with EGFP/pCIpA₁₀₂ mRNA. The upper panel was obtained in normal light conditions using phase contrast, while the lower panel is an overlay of green and red filtered photos of the same cells after excitation of EGFP and propidium iodide (PI), respectively (PI was added to cells on ice prior to analysis for staining of dead cells). Cells containing both EGFP and PI appear as yellow.

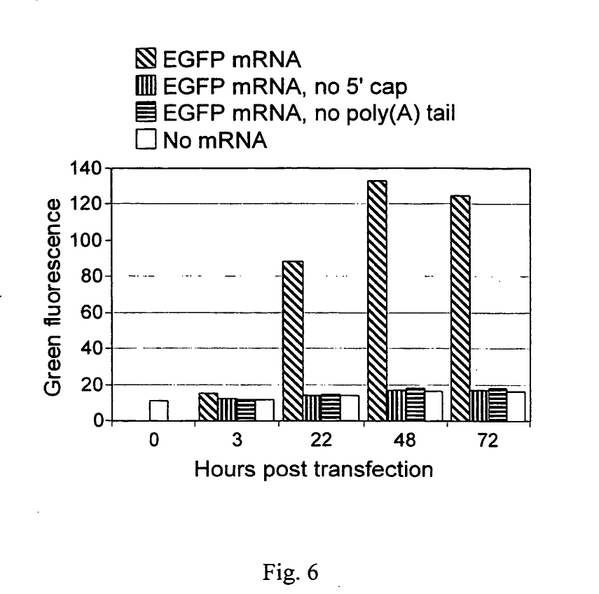

FIG. 6 is a graph illustrating electroporation of DCs with either EGFP/pCIpA₁₀₂ mRNA, EGFP/pCIpA₁₀₂ mRNA synthesised without cap analogs, or EGFP/pCI mRNA lacking a poly(A) tail. The cells were electroporated for 0.25 milliseconds at ˜2.4 kV/cm with 50 μg/ml RNA and monitored for accumulation of EGFP by flow cytometry. Each series represent mean values of two parallel experiments which produced almost identical results.

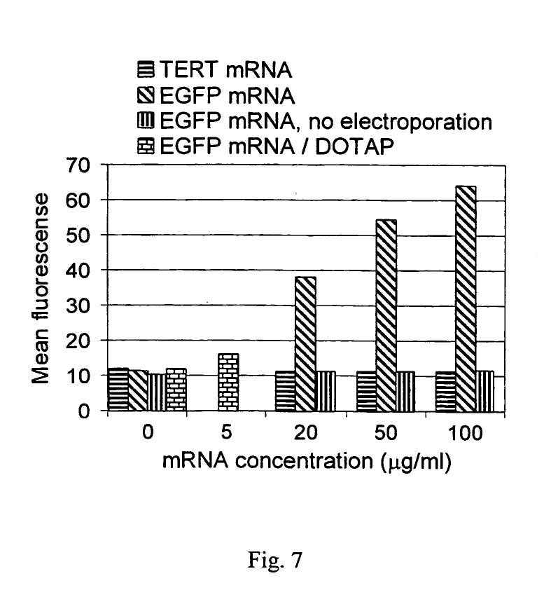

FIG. 7 is a graph showing a comparison between electroporation, sensitization (plain incubation) and liposome-mediated transfection of DCs with different concentrations of EGFP/pCIpA₁₀₂ mRNA. Electroporation was performed at ˜2.4 kV/cm for 0.25 ms. Sensitization and liposome-mediated transfection was carried out by mixing cells with mRNA or mRNA/DOTAP complex (1:5 ratio w/w), respectively, in RNase-free medium and incubating for two hours at 37° C. before seeding in complete medium. Cells were then incubated over night and analysed by flow cytometry.

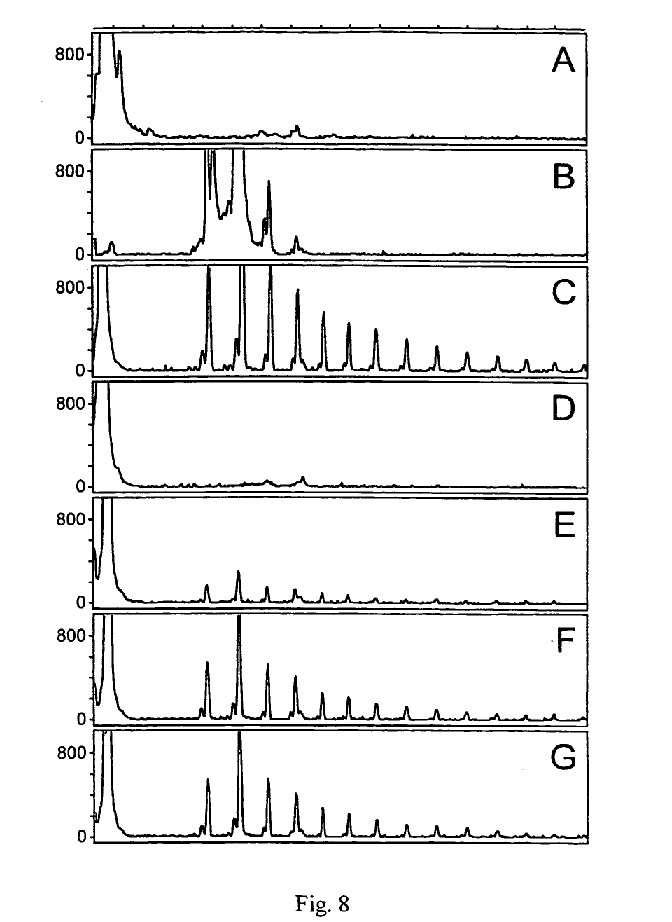

FIG. 8 is a graph illustrating induction of telomerase activity in DCs after transfection with mRNA encoding the telomerase catalytic subunit (hTERT). Cells were electroporated with hTERT/pCIpA₁₀₂ mRNA at a concentration 50 μg/ml and monitored for induction of telomerase activity using the TRAP assay. Each panel represent the equivalent of 1000 cells. The different panels show: A, mastermix; B, positive control assay using 1 attomol of a synthetic telomere consisting of the TS primer elongated with four telomeric repeat units; C, positive control assay using the HL60 cell line; D-G, DCs transfected with hTERT/pCIpA₁₀₂ mRNA and analysed 0 (D), 6 (E), 24 (F) or 48 (G) hours after transfection.

FIG. 9 shows poly(A-T) chains generated by PCR techniques and separated on polyacrylamide gels. The chains were synthesised by using either high-temperature PCR with the thermostable polymerase Pfu (lane 1-3), or low-temperature PCR with the non-thermostable large Klenow fragment of E. coli DNA polymerase I (lane 4-5). The temperature profiles used in the different lanes are: 1) 25×[75° C. for 1 sec.; 20° C. for 1 sec.; 72° C. for 5 sec.]; 2) 25×[75° C. for 1 sec.; 20° C. for 1 sec.; 72° C. for 5 sec.]; 3) 25×[20° C. for 1 sec.; 70° C. for 10 sec.]; 4) 25×[20° C. for 1 sec.; 37° C. for 5 sec.]; 5) 25×[40° C. for 5 sec.; 20° C. for 1 sec.; 37° C. for 5 sec.]. “M” indicates lane with molecular weight marker.

### EXAMPLES

Introduction

For the production of mRNA in vitro we have developed a panel of plasmid vectors, including a pBNco shuttle vector which is useful for construction of DNA transcription units from PCR products (FIG. 1), and several pCIpA mRNA expression vectors which contain poly(A) regions of different lengths as part of the inherent transcription unit (FIG. 2). These vectors can be used in combination, or separately, to produce capped mRNA with uniformly long poly(A) tails comprising up to several hundred nucleotides. In a transfection regimen combining this mRNA with electroporation, efficient reporter-gene expression can be induced in up to 100% of the cells. Mean reporter-gene expression and survival rate obtained in the cell population is dependent on the specific settings used for electroporation, and these conditions can be adjusted to meet different requirements. A 5′ cap and a long poly(A) tail were used for these constructs (FIG. 6), but other stabilising elements, such as the untranslated regions from the -globin RNA, can also be used.

We have discovered that the mRNA yield obtained with the Ribomax kit can be increased at least five-fold by adjusting the concentrations of rGTP and cap analogue (see Materials and Methods). By using this high concentration of mRNA the protocol became surprisingly efficient, even when compared to existing methods using plasmid DNA. Reporter-gene product levels resulting from mRNA-mediated transfection using mRNA with a 102-nt long poly(A) tail (pCIpA₁₀₂) generally reaches a maximum two days after transfection (FIG. 4B/C). When EGFP is used as a reporter mRNA (EGFP/pCIpA₁₀₂ mRNA), the mean EGFP signal obtained in DCs are 77× background level (BG), and with the strongest expressing cells at 400×BG. The highest obtainable transfection of DCs with EGFP/pCI plasmid DNA is achieved with square-wave electroporation at 2.5 kV/cm for 0.25 ms using 50 μg/ml DNA, and produces a mean EGFP signal within the sub-population having positive expression of 86×BG, with the strongest expressing cells at 1000×BG. However, since the transfection rate achieved with DNA is lower, the mean EGFP signal of the whole population is also lower, at 29×BG. Thus, with respect to the total EGFP expression obtained in the DC population, EGFP/pCIpA₁₀₂ mRNA is at least 2.6 times more efficient than EGFP/pCI plasmid DNA for electroporation.

We have compared electroporation with alternative mRNA-based transfection techniques presented in the literature, including sensitization (plain incubation with mRNA) and liposome-mediated transfection with DOTAP (FIG. 7). These alternative methods are not very efficient for mRNA-mediated transfection of DCs, and more than 200 times higher transfection efficiency can be achieved by electroporation (FIG. 4/7).

mRNA-based electrotransfection can be used with many, probably most, cell types, and for any purpose or experiment where a transient expression is required or sufficient. For example, we have employed the method to induce telomerase activity in DCs. DCs were transfected with full-length hTERT/pCIpA₁₀₂ mRNA by electroporation and the induction of telomerase activity was monitored using the TRAP assay (FIG. 8). The cells acquired telomerase activity in a time-based manner, and the enzymatic activity was approx. 40% of that in proliferating HL60 cells. By applying this treatment periodically, dividing cells can be kept in an immortalised state without having to be genetically modified on a permanent basis.

Materials and Methods

pBNco Shuttle Vector

The pBNco shuttle vector (FIG. 1) was constructed to allow cloning of blunt-end gene fragments is in the context (immediately downstream) of a consensus primate translation start sequence while at the same time offering the advantage of blue/white colour selection in cloning experiments. A DNA fragment containing cohesive SpeI and NotI ends, respectively, and internal NheI, NcoI and XbaI restriction cut sites, was made by hybridising two oligonucleotides (5′-CTAGTGCTAGCCACCATGGAGCTAGTTCTAGAGC; 5′-GGCCGCTCTAGAACTAGCTCCATGGTGGCTAGCA) and inserted between the SpeI and NotI sites of pBluescript SK(−) (Stratagene, La Jolla, Calif.; GenBank acc.: 52324). The inserted fragment adds the NheI and NcoI cut sites to the pBluescript SK(−) multiple cloning site (MCS), and is continuos with the lacZ open reading frame (ORF) without impairing normal lacZ function. Prior to use in cloning operations, pBNco was digested with NcoI followed by heat inactivation and a fill-in reaction of recessed 3′ ends using the large Klenow fragment of E. coli DNA polymerase I (DNApol I Klenow; NEB Inc., Beverly, Mass.) to generate a GCCACCATG-3′ blunt end for in-frame fusion. After inactivation of the polymerase the DNA was digested with XbaI or NotI to generate a downstream cloning site. The DNA was then de-phosphorylated with shrimp alkaline phosphatase (SAP; Roche, Basel, Switzerland) and purified with the Wizard PCR Preps kit (Promega Corp., Madison, Wis.) to generate the pBNco ready-for-cloning fragment (pBNco-R).

pCIpA₁₀₂ Expression Vector

For production of polyadenylated mRNA by in vitro run-off transcription, the pCI expression vector (Promega) was modified to contain a poly(A) region as part of its transcription unit. Poly(A-T) fragments were generated from d(A₂₀) and d(T₁₅) oligonucleotides by repeated synthesis (24 cycles: 40° C. for 5 s, 20° C. for 5 s, 37° C. for 5 s) on a PTC-200 thermal cycler (MJ Research, Waltham, Mass.) using DNApol I Klenow under proper reaction conditions (10 mM Tris-HCl pH 7.5; 5 mM MgCl₂; 7.5 mM DTT; 1 μM d(A₂₀) and d(T₁₅); 0.5 mM dATP and dTTP; 0.25 U/μl DNApol I Klenow), and inserted into the HpaI cut site in the SV40 3′UTR (untranslated region; designated by Promega as late polyadenylation signal) in pCI. The HpaI site is followed by a MunI/MfeI cut site which can be used to linearize the plasmid prior to in vitro transcription. A plasmid containing a 100-nucleotide long poly(A-T) insert, designated pCIpA₁₀₂ (FIG. 2), was chosen for further work.

EGFP/pCI and EGFP/pCIpA102 Constructs

The enhanced green fluorescence protein (EGFP) gene was isolated from pEGFP-N3 (Clontech, Palo Alto, Calif.) by digestion with EcoRI and NotI and inserted between the same cut sites in the pCI and pCIpA₁₀₂ expression vectors to generate EGFP/pCI and EGFP/pCIpA₁₀₂, respectively. Digestion of these constructs with MfeI and in vitro transcription using T7 RNA polymerase produce transcripts that contain (from 5′ to 3′): a 58-nt long 5′ UTR (mainly pEGFP-N3 polylinker), the EGFP ORF, the SV40 3′ UTR, a 102-nt long poly(A) tail (EGFP/pCIpA₁₀₂ only), and 14 nts of vector sequence.

hTERT/pCIpA₁₀₂ Construct

A plasmid construct containing the entire coding sequence (CDS) of the hTERT cDNA (formerly hEST2/hTRT) cloned in pCI-Neo (Promega) was kindly provided to us as a gift of Prof. Robert A. Weinberg, MIT, Cambridge, Mass. The CDS of this clone, except for the ATG start codon, was amplified by PCR using Pfu Turbo (Stratagene) and two suitable primers: a phosphorylated plus-strand primer (P-5′-CCGCGCGCTCCCCGCTGC) and a downstream primer (5′-GGTTTGTCCAAACTCATCAA) which hybridises within the SV40 3′ UTR in pCI-Neo. The PCR product was digested with NotI to remove the pCI-Neo vector sequence and ligated to the pBNco-R vector fragment. From this clone a NheI/NotI fragment containing the hTERT CDS was isolated and inserted between the respective cut sites in pCIpA₁₀₂ to generate hTERT/pCIpA₁₀₂. In vitro transcription of this construct with T7 RNA polymerase produces a transcript that contains (from 5′ to 3′): an 11-nt long 5′ UTR (5′-GGCUAGCCACC), the hTERT CDS, the SV40 3′ UTR, a 102-nt long poly(A) tail, and 14 nts of vector sequence.

Production of mRNA in vitro

Plasmid constructs were linearized with MfeI and purified by using phenol:chloroform:isoamyl alcohol (25:24:1; pH 7.8) extraction, chloroform:isoamyl alcohol (24:1) extraction, and ethanol precipitation. They were then transcribed in vitro using the Ribomax-T7 RNA production system (Promega) with the addition of m⁷G(5′)ppp(5′)G cap analogs (NEB). The reaction mix contained: 80 mM HEPES pH 7.5, 24 mM MgCl₂, 2 mM spermidine, 40 mM DTT, 7.5 mM rATP/rCTP/rUTP, 2.4 mM rGTP, 12 mM m⁷G(5′)ppp(5′)G, 0.1 mg/ml DNA template and 10% (v/v) enzyme mix (T7). After extraction with phenol:chloroform:isoamyl alcohol (25:24:1; pH 4.3) and chloroform:isoamyl alcohol (24:1), the mRNA was precipitated and washed with ethanol and dissolved in RNase-free water. The quality of the synthesized mRNA was checked by denaturing agarose/formaldehyde gel electrophoresis, and by binding to magnetic oligo(dT) Dynabeads (Dynal AS, Oslo, Norway).

Telomerase Assay

The protocol used to measure telomerase activity was modified from the Telomeric Repeat Amplification Protocol (TRAP; Kim, N. W. et al., 1994, Science 266: 2011-2015). CHAPS extracts were prepared from 10⁵ cells. The cells were washed first in PBS, then in HEPES wash (10 mM HEPES pH 7.5; 1,5 mM MgCl₂; 10 mM KCl; 1 mM DTT), and pelleted at 4° C. The cells were lysed by dissolving the pellet in 0.2 ml ice-cold CHAPS buffer (10 mM Tris pH 7.5; 1 mM MgCl₂; 1 mM EGTA pH 8.0; 0.1 mM benzamidine; 5 mM β-mercaptoethanol; 0.5% CHAPS; 10% glycerol) and incubated on ice for 30 minutes. The lysate was then centrifuged at 12000 g; 4° C. for 30 minutes, and the CHAPS extract (supernatant) was withdrawn and stored at −80° C. when not in use. Telomerase activity was measured by combining 2 μl CHAPS extract (equivalent to 1000 cells) with 48 μl master mix [200 nM TS primer, 40 nM fluorochrome-labelled CXA primer (HEX-5′-GTAGCCGCGCTTACCCTTACCCTTACCCTAACC), 20 mM Tris pH 8.0, 1.5 mM MgCl₂, 63 mM KCl, 1 mM EGTA, 0.005% Tween-20, 50 μM dATP/dCTP/dGTP/dTTP, and 0.05 U/μl Pfu Turbo]. The reaction mix was incubated for 10 min at 30° C., followed by 28 cycles of PCR (94° C. for 1 min, 50° C. for 1 min, 72° C. for 1 min), and a portion was then diluted 1:12 in formamide/size standard and analysed with the ABI prism 310 capillary electrophoresis unit (PE Corp., Norwalk, Conn.).

Preparation of Human Blood Cells

Buffy coats from normal HLA-A2⁺ donors were separated by density gradient centrifugation over Lymphoprep (Nycomed, Oslo, Norway), and the peripheral blood mononuclear cells (PBMCs) were isolated and cryopreserved in aliquots for later use as stimulators and responder cells. DCs were generated by plating thawed PBMCs in 6-well plates at 10⁷ cells/well in X-VIVO 10 (BIO-Whittaker, Walkersville, Md.) supplemented with 2% heat-inactivated human pool serum. The cells were allowed to adhere for 1.5 hrs in 5% CO₂ at 37° C., and the non-adherent cells were removed. The adherent cells were washed three times and suspended in X-VIVO 10 supplemented with 2% heat-inactivated human pool serum, 800 U/ml GM-CSF, 500 U/ml IL-4, 10 ng/ml TNF-α and 100 U/ml INF-α (hereafter referred to as maturation medium), and incubated (5% CO₂ at 37° C.) 4-7 days for differentiation of DCs. The phenotype of differentiated cells was evaluated by staining with fluorochrome-labelled antibodies against the MHC-II, CD80, CD83, CD86, CD1a and CD14 cell surface markers and analysed by flow cytometry using the FACSCalibur flow cytometer (Becton Dickinson Immunocytometry Systems, San Jose, Calif.). Differentiation of mature DCs, as measured by up-regulation of MHC-II, CD80, CD83 and CD86, and down-regulation of CD1a and CD14, was complete on day 5 (results not shown).

Transfection of DCs

DCs were washed once and suspended in X-VIVO 10 and placed on ice. In the case of DNA transfection, 0.1 ml (10⁴-10⁵) cells were mixed with 2 μg DNA (1 μg/μl). When transfecting with mRNA, 0.2 ml (10⁵-10⁶) cells were mixed with 10-50 μg mRNA (1-5 μg/μl). The cells were transferred to a 2 mm-gap cuvette and pulsed with a BTX ECM 830 square-wave electroporator (Genetronics Inc., San Diego, Calif.) using parameter settings as specified in the text. After incubation on ice for one minute, the cells were seeded in maturation medium and incubated at 37° C. Cells transfected to express EGFP (EGFP/pCI plasmid, EGFP/pCIpA₁₀₂ mRNA) were analysed with the FACSCalibur flow cytometer. Expression of hTERT after transfection with hTERT/pCIpA₁₀₂ mRNA was analysed using the telomerase assay described above.

Induction of Primary CTL Responses and Cytotoxicity Assay

DCs were transfected with hTERT/pCIpA₁₀₂ mRNA and incubated for 24 hours in maturation medium. They were then washed and mixed with thawed autologous PBMCs at a stimulator to responder ratio of 1:10 in X-VIVO 10 supplemented with 10% heat-inactivated human pool serum and 5 U/ml recombinant interleukin-2 (rIL-2). The bulk cultures were restimulated weekly, and partial replacement of medium was done twice a week. Cytotoxicity assays were performed 10 days after the last restimulation. The induction of CTL responses after priming with hTERT was monitored in a conventional ⁵¹Cr-labelling release assay. DCs transfected with hTERT/pCIpA₁₀₂ mRNA, EGFP/pCIpA₁₀₂ mRNA, or just electroporated (the latter two for controls) were used as target cells. The cells were incubated with 7.5 MBq ⁵¹Cr in a total volume of 0.5 ml at 37° C. for 1 h, then washed three times, and seeded in 96-well U-bottomed microtitre plates (Costar, Cambridge, Mass.) at 2×10³ cells/well. 2×10⁴ effector cells were added to each well, and the plates were incubated 4 hrs at 37° C. Supernatants were then harvested, and radioactivity was measured in a Topcount microplate scintillation counter (Packard Instrument Company Inc., Meriden, Conn.). Maximum and spontaneous ⁵¹Cr release was measured after incubation with 5% Triton X-100 or medium, respectively. Specific release was calculated by the formula: (experimental release-spontaneous release)/(maximum release-spontaneous release).

Optimized Conditions for Transfection of DCs by Square-wave Electroporation

To determine the functional requirements for transfection of DCs by square-wave electroporation, cells were transfected with EGFP/pCI plasmid DNA using various combinations of voltage and pulse length. In contrast to transfection with mRNA, which is translated into protein immediately after entering the cytoplasm, plasmid DNA must also traverse the nuclear envelope in order to be expressed. This is an inefficient process and only a minor fraction (in the range 10⁻³-10⁻⁴) of the plasmid DNA entering a cell becomes available for expression in the nucleus. Nonetheless, since the amount of DNA entering the nucleus depends directly on its cytosolic concentration it is reasonable to assume that the optimal requirements for these transfection methods are similar. In this example we used the EGFP/pCI plasmid to analyse the relationship between transfection efficiency and specific voltage and time settings used with the ECM830 square-wave electroporator. DCs were harvested, washed, and suspended in ice cold medium. For each transfection, 0.1 ml (10⁴) cells were mixed with 2 μg DNA (1 μg/μl) and pulsed in a 2 mm-gap cuvette using different combinations of voltage and pulse length. After incubation for two days in complete medium the cells were analysed by flow cytometry. As shown in FIG. 3, DCs can be efficiently transfected by square-wave electroporation using a wide range of different instrument settings. Optimal transfection of DCs was achieved when applying a voltage (as reported by the instrument) in the range 1.5-3 kV/cm (kilovolt/cm) for 0.15-0.25 ms (milliseconds), and with a maximum peak at 2.5 kV/cm for 0.25 ms producing 15% transfected cells. Considerable transfection was also obtained both at higher voltages/shorter pulses and lower voltages/longer pulses, whereas long-lasting pulses (several milliseconds or more) in combination with low voltage was inefficient and/or deleterious to the cells. The requirements for electroporation of DCs are thus considerably different from those required by cell lines and many other cell types which generally involve a combination of low voltage and a long-lasting pulse (a compilation of current protocols can be obtained from the BTX homepage at http://www.btxonline.com/btx/index.html). The broad window of different instrument settings which produce efficient transfection allows for adaptation to different experimental requirements by balancing between transfection efficiency and cell survival. Several modifications to the electroporation buffer were also tested and found to improve transfection rates to those shown in FIG. 3 when applied in moderate amounts, including increased concentration of DNA, reduced concentration of overall salt, and exchange of NaCl with potassium salts. The latter modifications also had a positive effect on survival rates (results not shown).

To test whether square-wave electroporation also can be applied to other cell types, we performed a similar screening of parameters with bone marrow-derived CD34+ stem cells. Although these cells showed to be less tolerant to extended pulse lengths, considerable transfection and good survival rates (50-90%) was achieved when pulsed at 2.2-4 kV/cm for 0.03-0.1 ms. Highest transfection efficiency was obtained with 3.5 kV/cm for 0.05 ms and yielded 7.9% transfected cells. We have also observed good transfection of lymphocytes, which often accompanies monocyte-derived DCs isolated by adherence to plastic. When present, these were efficiently transfected along with the DCs. Thus, square-wave electroporation may serve as an alternative for transfection of many cell types.

mRNA-based Transfection of Primary Cells by Electroporation

The easy access of transfected mRNA to the cytoplasmic translation machinery and its limited ability to cause permanent genetic changes makes it an attractive approach for transient transfection of cells. However, previous strategies for mRNA-mediated transfection have been very ineffective compared to alternative transfection methods, and for most applications they have not represented an alternative. To overcome these problems we have established a transfection method which uses square-wave electroporation for transfection of mRNA into cells. The mRNA can be isolated directly from cell samples, such as a tumour biopsy, or it can be synthesised in vitro by run-off transcription. For the latter purpose we have developed two plasmid vectors, a pBNco shuttle vector (FIG. 1) and a pCIpA₁₀₂ mRNA expression vector (FIG. 2), which allows for efficient cloning of constructs and production of mRNA with uniform 102-nt long poly(A) tails. Preliminary evaluation of the method was performed by cloning the enhanced green fluorescence protein (EGFP) in pCIpA₁₀₂ for subsequent production of capped mRNA according to the procedure described in the Ribomax-T7 kit manual. DCs were electroporated with ˜50 μg/ml mRNA using different voltage settings and the expression profile following transfection was monitored. As shown in FIG. 4A-B, this mRNA-mediated electroporation was able to efficiently transfect the entire DC population (95-100%) both at moderate and forceful electroporation conditions, with the mean EGFP signal in the population being positively correlated with the applied voltage (FIG. 4A). Following transfection, the mean signal increased steadily and reached a peak value at 12×BG (background level) by approximately 48 hours (FIG. 4B), at which expression and degradation of EGFP is balanced out. During the next three days the mean EGFP signal decreased with 35%. However, the expression profile in this phase is strongly influenced by the half-life of the particular protein being expressed and may progress differently with other proteins.

We have discovered that the mRNA yield obtained with the Ribomax kit can be increased at least five-fold by adjusting the concentrations of rGTP and cap analogue (see Materials and Methods). To test whether this modification is beneficial with regard to overall transfection efficiency, DCs were electroporated with an equivalent volume of reaction product of this mRNA as for that used in FIG. 4B, resulting in a final mRNA concentration of ˜250 μg/ml. The expression profile was monitored, and as shown in FIG. 4C, the modified procedure produced an increase in mean EGFP fluorescence that is roughly proportional to the increased concentration of EGFP mRNA, indicating that mRNA quality (capping efficiency) is preserved. By using this high concentration of mRNA the protocol also became remarkably efficient. After 38 hours of incubation, which represent the peak value of the measurements undertaken, the mean EGFP signal reached 77×BG, and with the strongest expressing cells at 400×BG. Five days after transfection the cells still appeared as bright shining stars under the microscope (FIG. 5). At this stage a fraction of the cells was observed to absorb propidium iodide from the medium, indicating leakiness and lowered survival rates. This is probably, at least in part, due to toxic effects from high intracellular EGFP concentrations, as these cells show strong EGFP staining in the nucleus (appearing with yellow colour).

mRNA-mediated electroporation is also very efficient when compared to methods using plasmid DNA. The highest obtainable transfection of DCs in our system with EGFP/pCI plasmid DNA was achieved with square-wave electroporation at 2.5 kV/cm for 0.25 ms using 50 μg/ml DNA. After 48 hours of incubation the sub-population expressing EGFP had a mean EGFP signal at 86×BG with the strongest expressing cells at 1000×BG. However, due to the lower transfection rate achieved with DNA, the mean EGFP signal of the whole population of surviving cells is lower, at 29×BG. Thus, with respect to the total EGFP expression obtained in the DC population, mRNA was 2.6 times more efficient than plasmid DNA for electroporation.

The 5′ cap and poly(A) tail of mRNA is recognised as important factors for translation initiation and stability (Tarun, S. Z. et al., 1997, Proc Natl Acad Sci USA 94: 9046-9051). To test the importance of these factors in our system, standard electroporation with EGFP/pCIpA₁₀₂ mRNA was compared to electroporation with EGFP/pCIpA₁₀₂ mRNA synthesised without cap analogs and EGFP/pCI mRNA lacking a poly(A) tail. As shown in FIG. 6, no significant expression occurred when omitting either of these elements, and hence, the 5′ cap and poly(A) tail is essential for expression of these mRNA constructs. We also compared electroporation with alternative mRNA-based transfection techniques presented in the literature, including sensitization (plain incubation with mRNA) and liposome-mediated transfection with DOTAP (FIG. 7). Sensitization was performed by incubating DCs with EGFP/pCIpA₁₀₂ mRNA in RNase-free medium for two hours before addition of complete medium, but even at the highest concentration of mRNA applied (100 μg/ml), this treatment produced no significant increase in fluorescence levels. We also performed a series of experiments with EGFP/pCIpA₁₀₂/DOTAP complexes, but discovered serious limitations of using this treatment with DCs due to toxicity. Best transfection was achieved by transfecting cells for two hours in RNase free medium with 5 μg/ml EGFP/pCIpA₁₀₂ mRNA aggregated with DOTAP in a ratio of 1:5 (w/w). This treatment killed 85% of the DCs and the mean fluorescence of the remaining cells was increased by 36% (1.36×BG) compared to non-transfected cells. In summary, these alternative methods were not very efficient for mRNA-mediated transfection of DCs, and at least 200 times (77×BG−1/1.36×BG−1) higher transfection efficiency could be achieved by electroporation.

The high transfection efficiencies accomplished with mRNA-based square-wave electroporation offers a convenient method for direct transfection of tumour derived mRNA into DCs for stimulation of cellular immune responses, and may eliminate the need for prior amplification steps.

Induction of hTERT-specific Cytotoxic T Lymphocytes

The telomerase catalytic subunit (hTERT) is a component of the telomerase complex and plays a key role in the maintenance of genome stability by adding telomeres to the ends of linear chromosomes. Telomerase activity is normally expressed in germinal tissues and early embryos, and has also been detected in some proliferating adult tissues including bone marrow stem cells (Yui, J. et al., 1998, Blood 9: 3255-3262; Uchida, N. T. et al., 1999, Leuk Res 23: 1127-1132), epithelial cells in colonic crypts (Tahara, H. et al., 1999, Oncogene 18: 1561-1567) and activated lymphocytes (Liu, K. et al., 1999, Proc. Natl. Acad. Sci. USA 96: 5147-5152; Son, N. H. et al., 2000, J. Immunol. 165: 1191-1196). Most adult tissues do not contain telomerase activity (reviewed in Dhaene, K. et al., 2000, Virchows Arch 437: 1-16), and since the telomerase complex is subject to various modes of regulation, the absence of telomerase activity may have different explanations. The hTERT gene can be transcriptionally inactive, a functional complex may be present but inhibited by other factors, or the hTERT pre-mRNA may be spliced to yield non-functional variants. However, reactivation of telomerase activity appears to be a prerequisite for immortalization and malignant transformation, and has been detected in most human cancers. This has triggered a debate as to whether the hTERT protein may serve as a target for cancer immunotherapy, and a few reports have emerged supporting this notion (Minev, B. et al., 2000, Proc. Natl. Acad. Sci. USA 97: 4796-4801; Nair, S. K. et al., 2000, Nature Medicine 6: 1011-1017).

To test the legitimacy of this proposition in a system with high expression of hTERT and naturally processed epitopes, which is the situation in proliferating cancer cells, DCs were transfected with full-length hTERT/pCIpA₁₀₂ mRNA by electroporation for stimulation of autologous T cells. Verification of transfection efficiency was performed by monitoring the induction of telomerase activity using the TRAP assay. As shown in FIG. 8, the cells acquired telomerase activity in a time-based manner, and after 24 hours the activity was approx. 40% of that in proliferating HL60 cells. A total of 10 buffy coats from different HLA-A2⁺ donors were processed as outlined in Materials and Methods and PBMCs were stimulated weekly with hTERT-positive DCs. After the fourth stimulation, testing of toxicity was performed with three randomly selected samples in a conventional ⁵¹Cr-labelling release assay. Specific release with autologous hTERT-positive DCs as target cells was 6, 17 and 33%, respectively, while the negative controls with EGFP-positive DCs as targets were all negative. We also processed four buffy-coats by using an alternative protocol where monocytes were differentiated to immature DCs by incubation with GM-CSF and IL-4, transfected on day 10, and then incubated for two days in maturation medium prior to stimulation of T cells.

Differentiation of a mature DC phenotype was verified by immuno-staining (see Materials and Methods) and was not affected by electroporation, when compared to non-transfected cells. In this regimen, T cell responses developed faster, and after the third stimulation specific lysis of hTERT-loaded DCs was above 100% (compared to lysis with 5% Triton-X) for all cultures.

The experiments show that mRNA-based electroporation can be used for loading of DCs and induction of T cell responses, and that it is very effective in doing so.

PCR-based Synthesis of Poly(A-T) Chains

The present invention utilises expression vectors containing long poly(A-T) chains [one strand is poly(A) and its complementary strand is poly(T)] for production of polyadenylated mRNA. A method for producing long poly(A-T) chains was developed using PCR with oligo d(A) and oligo d(T) oligonucleotides. The oligonucleotides serve both as primers and template in the PCR reaction, and the poly(A-T) chains synthesised grow longer with each PCR cycle until they reach a maximum length in which the melting point of the formed DNA duplex is higher than the temperature used in the denaturation (or combined denaturation/synthesis) step. Thus, the poly(A-T) length obtained depend on general PCR parameters like melting temperature and number of cycles, and in particular on whether the PCR is a conventional high-temperature PCR or performed at lower temperatures using a non-thermostable DNA polymerase.

Poly(A-T) synthesis by high-temperature PCR was performed in a reaction containing 1 μM oligo d(A₂₀), 1 μM oligo d(T₁₅), 0.5 mM dATP, 0.5 mM dTTP, 1×Pfu buffer and 0.06 U/μl Pfu DNA polymerase (Stratagene). For low-temperature PCR the reaction contained 1 μM oligo d(A₂₀), 1 μM oligo d(T₁₅), 0.5 mM DATP, 0.5 mM dTTP, 10 mM Tris-HCl pH 7.5, 5 mM MgCl₂, 7.5 mM DTT and 0.25 U/μl DNApol I Klenow (NEB). The PCRs were run on a PTC-200 thermal cycler (MJ Research), and detailed description of some illustrative temperature profiles and results is given in FIG. 9. Poly(A-T) chains produced by high-temperature PCR ranged from 100 to 10000 bp in length, and the average length could be adjusted by modifying PCR parameters. Low-temperature PCR produced shorter poly(A-T) chains of more uniform length, ranging mainly from 100-200 bp, plus a smaller fraction of longer chains ranging up to approximately 2500 bp. Ten chains in the range 100-300 bp were sequenced after insertion into the pCI vector and all confirmed to a poly(A-T) configuration, being either homogenous poly(A) or poly(T) on the strand sequenced. Thus, PCR techniques using d(A) and d(T) oligonucleotides as primer/template is an efficient method for production of long poly(A-T) chains.

1. In a method of transfecting RNA into a cell that includes the step of forming a mixture comprising the RNA, the cell, and a suspension medium and the step of applying one or more electrical pulses to the mixture, the improvement wherein the RNA that is chosen has a poly(A) tail.

2. (Cancelled)

3. A method according to claim 1, wherein the poly(A) tail is at least 70 nucleotides long.

4. A method according to claims 1 or 3, wherein the RNA has a 5′ cap.

5. A method according to claim 4, wherein the RNA is mRNA.

6. A method according to claim 4, wherein the RNA is a tumor cell RNA.

7. A method according to claim 4, wherein the RNA encodes all or part of a telomerase.

8. A method according to claim 1, wherein the RNA is introduced into the cell by electroporation.

9. A method according to claim 8, wherein the electroporation is square wave electroporation.

10. A method according to claims 1, 8 or 9, wherein one or more pulses lasting from 0.0001 to 100,000 milliseconds at a voltage of from 0.001 to 1000kV/cm are used.

11. (Cancelled)

12. A method according to claim 1, wherein the cell is a primary cell.

13. A method according to claim 1, wherein the cell is an antigen presenting cell.

14. A method according to claim 1, wherein the method achieves at least 10% transfection of the cells in the mixture.

15. The method according to claim 1, wherein the method is used in the preparation of a cell for cell-based therapy.

16. The method according to claim 1, wherein the method is used in the preparation of a cell for immunotherapy.

17. In a process of preparing a vaccine that contains a transfected cell that is prepared by a method of transfecting RNA into a cell that includes the step of forming a mixture comprising the RNA, the cell, and a suspension medium and the step of applying one or more electrical pulses to the mixture, the improvement wherein the RNA that is chosen has a poly(A) tail.

18. In a process of preparing an anticancer vaccine that contains a transferred cell that is prepared by a method of transfecting RNA into a cell that includes the step of forming a mixture comprising the RNA, the cell, and a suspension medium and the step of applying one or more electrical pulses to the mixture, the improvement wherein the RNA that is chosen has a poly(A) tail.

19. The invention as substantially hereinbefore described, with reference to the accompanying drawings and examples.

20. The method according to claim 10, wherein one or more pulses lasting from 0.01 to 1000 milliseconds is used.

21. The method according to claim 10, wherein a voltage of from 0.1 to 10 kV/cm is used.

22. The method according to claim 12, wherein the primary cell is selected from the group consisting of lymphocyte cells, stem cells and dendritic cells.

23. In a method of transfecting RNA into a cell that includes the step of forming a mixture comprising the RNA, the cell, and a suspension medium and the step of applying one or more electrical pulses to the mixture, the improvement wherein the RNA that is chosen has a 5′ cap and a poly(A) tail, the poly(A) tail is at least 70 nucleotides long, the RNA is introduced into the cell by square wave electroporation, one or more pulses lasting from 0.0001 to 100,000 milliseconds at a voltage of from 0.001 to 1000 kV/cm are used when applying the electrical pulse(s), and the cell is a primary cell selected from the group consisting of lymphocyte cells, stem cells and dendritic cells.
